(19) United States
(12) Patent Application Publication
(10) Pub. No.: US 2010/0063548 A1
(43) Pub. Date: Mar. 11, 2010

(54) Spinal Correction Method Using Shape Memory Spinal Rod

(75) Inventor: Yan Wang, Beijing (CN)

Correspondence Address: NUTTER MCCLENNEN & FISH LLP, SEAPORT WEST, 155 SEAPORT BOULEVARD, BOSTON, MA, 02210-2604, US

(73) Assignee: DePuy International Ltd, Leeds (GB)

(21) Appl. No.: 12/497,850

(22) Filed: Jul. 6, 2009

Related U.S. Application Data
(60) Provisional application No. 61/078,519, filed on Jul. 7, 2008.

(51) Int. Cl. A61B 17/88

(52) U.S. Cl. 606/279

(57) ABSTRACT

Various methods and devices related to correcting a spinal deformity are disclosed herein. In one embodiment, a method is provided and can include attaching to at least a portion of a patient's spine a spinal correction template having a first configuration, which substantially corresponds to an uncorrected shape of a spine. The spinal correction template can be activated such that the template achieves a second configuration to cause the spine to assume an orientation substantially corresponding to the second configuration of the spinal correction template. The method can further include attaching a primary spinal rod to at least a portion of a spine after the spine achieves a corrected orientation, and removing the spinal correction template. In some embodiments, the method can include inserting a secondary spinal rod in place of the spinal correction template. In one embodiment, the spinal correction template can be formed of a shape-memory alloy such as nitinol.

### RELATED APPLICATIONS

The present application claims priority to U.S. Provisional Application No. 61/078,519 filed on Jul. 7, 2008 and entitled “Spinal Correction Method Using Shape Memory Spinal Rod,” which is expressly incorporated herein by reference in its entirety.

### FIELD OF USE

Methods and devices are provided herein for use in spinal surgery, and in particular for use in correcting spinal deformities.

### BACKGROUND

Spinal corrective systems may be used in orthopedic surgery to correct a deformity or misalignment in the spinal column caused by disorders such as scoliosis, spondylosis, spondylolisthesis, as well as by injuries such as compression fractures. In spinal corrective surgery, spinal corrective devices, such as a spinal rod and anchoring devices, may be used to stabilize the spine and/or bring misaligned vertebrae back into alignment and secure the aligned vertebrae in the aligned position. A standard surgical procedure for correcting spinal defects in the current state of the art involves pulling the spinal column into alignment, and then stabilizing the spine using spinal corrective devices (e.g., rods) that are anchored to bone.

A spinal corrective device used in such systems is generally a relatively rigid fixation rod or plate that is coupled to a bone by way of various anchoring devices. The corrective device can extend between two or more bone regions to effect stabilization, positioning, and/or fixation of the bones. The spinal corrective device can have a predetermined contour that has been designed according to the properties of the target implantation site and, once installed, the spinal corrective device holds the bones in a desired spatial relationship, either until desired healing or spinal fusion has occurred, or for some longer period of time.

Current spinal corrective devices and techniques for using such devices rely on forcing displaced vertebral bodies into alignment, usually by manual force, such that a rigid rod can be attached to correct the deformity. Such techniques can be challenging for the surgeon and painful for the patient. A surgeon generally must make a compromise between the starting, deformed shape of the spine and the desired shape so that the spinal rods can be connected. The forces required to pull the vertebral bodies into alignment can be large, unevenly applied, and sometimes difficult to control. In addition, the space in which a surgeon has to work is small relative to the force required for aligning and holding the vertebrae in place while a spinal corrective device is applied. In addition, if only a few anchoring points are used in a particular procedure, there is a risk of screw pull out or damage to the screw head while the surgeon is trying to fit the rod profile to the deformed spine.

Accordingly, there is a need for methods and devices that would allow a surgeon to carry out a correctional maneuver with greater ease and accuracy.

### SUMMARY OF THE INVENTION

Disclosed herein are methods and devices related to correcting a spinal deformity. In one embodiment, a method is provided and can include attaching to at least a portion of a patient's spine a spinal correction template having a first configuration, which substantially corresponds to an uncorrected shape of a spine. The spinal correction template can be activated such that the template achieves a second configuration to cause the spine to assume an orientation substantially corresponding to the second configuration of the spinal correction template. The method can further include attaching a primary spinal rod to at least a portion of a spine after the spine achieves a corrected orientation, and removing the spinal correction template. In some embodiments, the method can include inserting a secondary spinal rod in place of the spinal correction template. While the spinal correction template can be formed of any materials known in the art, in one embodiment, the spinal correction template can be formed of a shape-memory alloy such as nitinol. In addition, the step of activating the spinal correction template can include heating the spinal correction template to a temperature above its activation temperature.

The primary spinal rod can have various configurations, and in one embodiment it can have a configuration substantially the same as the second configuration of the spinal correction template, i.e., a desired, corrected orientation of the spine. Likewise, the secondary spinal rod can have various configurations, and in one embodiment it can have a configuration substantially the same as the second configuration of the spinal template. The first configuration of the spinal correction template can substantially mimic the uncorrected shape of a deformed spine and the second configuration can substantially mimic a desired shape of the spine, such as the shape of a normal spine.

The spinal correction template can have various lengths suitable to provide the desired correction. In one embodiment, the spinal correction template can have a length configured to provide a twelve-level spinal correction. The spinal correction template can also have a length configured to provide at least a three-level spinal correction. In one embodiment, the step of placing the spinal correction template into the body can be performed using a minimally invasive surgical technique.

The spinal correction template can generally be attached to the spine at a temperature less than a temperature at which activation occurs. In some embodiments, the step of activating can occur at a temperature in the range of about 28 degrees Celsius to about 37 degrees Celsius. The step of attaching can occur when the spinal correction template is at a temperature of about 0 degrees Celsius. Heating of the spinal correction template can occur in various ways, for example simply by relying on heat transfer from a patient's body. Alternatively, heat can be derived from external sources, such as by the use of radiant heating, liquid heating, and electromagnetic heating.

Other methods are provided and can include a method for adjusting a curvature of a spine that includes determining an initial curvature shape and a final curvature shape for a patient's spine, providing a template having the final curvature shape, changing a temperature of the template to move the template to the initial curvature shape, attaching the template to the patient's spine in the initial curvature shape, adjusting the curvature of the patient's spine by changing the temperature of the template to cause it to move from the initial curvature shape to the final curvature shape. In some embodiments, providing a template can include providing a nitinol spinal rod. The method can also include applying a permanent spinal rod having the final curvature shape to the patient's spine and removing the template from the patient's spine. These and other aspects of the presently disclosed methods and devices are described in detail below.

In other aspects, a spinal correction kit is provided and can include a plurality of spinal anchors configured to be positioned within bone, each spinal anchor having a rod receiving portion. The kit can also include one or more spinal correction template configured to be positioned within the rod receiving portions in an initial orientation parallel with a patient's spine and to move the patient's spine to a desired orientation in response to an activation energy. The kit can further include one or more permanent spinal rods. In some embodiments, the spinal correction template can include a nitinol rod and the activation energy can include a change in temperature.

### BRIEF DESCRIPTION OF THE DRAWINGS

The various embodiments of the presently disclosed methods and devices will be more fully understood from the following detailed description taken in conjunction with the accompanying drawings, in which:

FIG. 1A is an illustration of one embodiment of a temporary spinal correction template in a rigid state;

FIG. 1B is an illustration of the spinal correction template of FIG. 1A in a flexible state and being bent into a desired shape;

FIG. 1C is an illustration of the spinal correction template of FIG. 1B maintaining the desired shape in the flexible state;

FIG. 1D is an illustration of the spinal correction template of FIG. 1A returning to its rigid state;

FIG. 2 is a side view of one embodiment of a retaining portion of an anchor having at temporary spinal correction template of FIG. 1 positioned therein;

FIG. 3A is a posterior view of a spinal correction template in a second configuration;

FIG. 3B is a posterior view of an exemplary deformed spine having anchors attached thereto;

FIG. 3C is a posterior view of the spinal correction template of FIG. 3A in a first configuration;

FIG. 3D is a posterior view of a spine having the spinal correction template of FIG. 3C in the first configuration;

FIG. 3E is a posterior view of the spine of FIG. 3D as the spinal correction template changes from the first configuration to the second configuration as heat is applied from an external source;

FIG. 3F is a posterior view of the spine of FIG. 3E in the second configuration, following correction of the deformed spine as a result of the spinal correction template changing from the first configuration to the second configuration;

FIG. 4A is a posterior view of the corrected spine of FIG. 3F having a permanent spinal rod attached thereto;

FIG. 4B is a posterior view of the corrected spine of FIG. 3F having the template removed;

FIG. 4C is a posterior view of the corrected spine of FIG. 3F having a secondary permanent spinal rod attached thereto;

FIG. 4D is a posterior view of the corrected spine of FIG. 3F having two permanent spinal rods attached thereto;

FIG. 5A is a perspective view of one embodiment of a spinal correction template in a first configuration and a deformed spine;

FIG. 5B is a perspective view of the template of FIG. 5A attached to the deformed spine;

FIG. 5C is a perspective view of the template of FIG. 5A in a second configuration having provided a correction to the deformed spine;

FIG. 6A is a perspective view of another embodiment of a spinal correction template in a first configuration and an injured spine;

FIG. 6B is a perspective view of the template of FIG. 6A attached to the injured spine; and

FIG. 6C is a perspective view of the template of FIG. 6A in a second configuration having provided a correction to the injured spine.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the presently disclosed methods and devices.

The present invention generally provides an improved surgical system, device, and method for correcting scoliosis, spondylolisthesis, spinal injuries, and other spinal deformities in a patient through the use of a temporary spinal correction template, which can be made from a shape memory alloy. In general, a spinal correction system is provided having a number of spinal corrective devices, at least one of which is formed from a shape memory material. For example, in one embodiment, the system can include a temporary spinal correction template formed of a shape memory material, at least one permanent spinal rod, and an appropriate number of anchoring devices. In other embodiments, the system can include a temporary spinal correction template and two permanent spinal rods, for example, a primary rod and a secondary rod, as well as anchoring devices. One skilled in the art will recognize that a spinal correction system according to the teachings of the invention may have any suitable number of spinal corrective devices, and that the dimensions of such rods can vary depending on factors such as the desired application and patient size.

In general, methods are also provided related to using a temporary correction template to provide an initial correction of a patient's spine through the use of a shape memory alloy. The method can generally include attaching to at least a portion of a patient's spine a spinal correction template having a first configuration (corresponding to the uncorrected shape of the spine), and activating the spinal correction template such that the template achieves a second configuration to cause the spine to assume the second configuration of the spinal correction template (corresponding to a desired shape of a corrected spine). The method can further include attaching a primary spinal rod to at least a portion of the spine and removing the spinal correction template following attachment of the primary spinal rod.

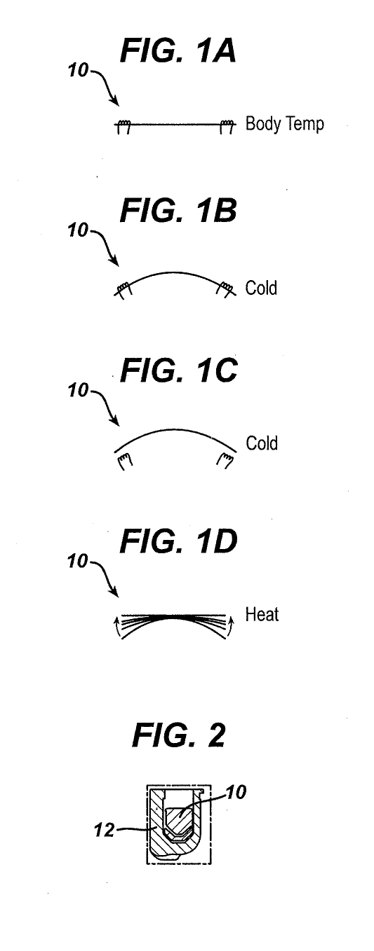

FIG. 1 illustrates an example of a temporary correction template 10 useful with the disclosed method, which in one embodiment, can be formed of or can include a shape memory material having pseudoelastic or superelastic properties. For example, the shape memory material can be a shape memory polymer or alloy, such as nitinol (a nickel-titanium alloy), to allow for a change in shape that can create corrective forces on the spine. A shape memory material is generally characterized by having two distinct states and an ability to restore itself to a preselected, predetermined, or preconfigured shape of a particular state after plastic deformation. For example, in an austenitic state (e.g., above its activation temperature), the shape memory material is stiff, rigid and has a set, preselected shape. In a martensitic state (e.g., below its activation temperature), the shape memory material becomes flexible and deformable and may be caused to assume any of a variety of shapes. That is, in the martensitic state, the material can be shaped as desired. Once the material is returned to its austenitic state, e.g., by heating to above its activation temperature, it will “automatically” return to its original, preselected shape. In general, the microstructure of the material in the martensitic state is characterized by “self-accommodating twins,” having a zigzag arrangement, which allow for deformation of the material shape by de-twinning.

In one embodiment in which the temporary correction template 10 is formed of a shape memory material, the template 10 can become flexible in response to an activation energy (e.g., by cooling it below its activation temperature) and can be easily bent into a variety of different shapes, allowing the template 10 to be fitted to a deformed spine. In the course of transitioning to an austenitic state in response to an activation energy, e.g., by heating it to above its activation temperature, the template 10 can achieve a stiff, high-strength structure to thereby apply corrective forces to a deformed spine. In general, shape memory materials can be induced to transition between the rigid austenitic state and the flexible martensitic state by changing the temperature, pressure, stress, chemistry and/or another parameter of the shape memory material.

While devices and methods provided herein can be effective to activate a shape memory material transition between martensitic and austenitic states by any one of the mechanisms noted above, in one exemplary embodiment, the transition can be activated using thermal techniques such as a temperature change. For example, the temporary correction template 10, formed of a shape memory material, can be cooled to make the material flexible in a martensitic state, and subsequently heated to return the template 10 to a preselected shape having the austenitic structure. When the template 10 is in the martensitic state, e.g., a first configuration, it can be returned to a preselected shape, e.g., a second configuration, of the austenitic state by changing the temperature of the template 10. In one embodiment, changing the temperature of the template 10 can mean raising the temperature of the template 10 above a preselected temperature. In other embodiments, changing the temperature of the template 10 can mean lowering the temperature of the template 10 below a preselected temperature.

In general, an activation temperature of the correction template 10 can refer to a temperature range at which the template 10 begins transforming to austenite and thus changing its shape to return to a second configuration. More particularly, the temperature at which a shape memory material starts transforming to austenite is known as the “austenite start temperature.” Further heating of the material increases the temperature of the shape memory material to induce a complete transformation to the austenitic state. The temperature at which a shape memory material finishes transforming to austenite is known as the “austenite finish temperature.” Likewise, a shape memory material can transition to the martensitic state to allow deformation and shaping of the material by changing the temperature of the material below a selected temperature (i.e., cooling the material). The temperature at which a shape memory material begins transformation to the martensite state is known as the “martensite start temperature.” Further cooling decreases the temperature of the shape memory material to induce a complete transformation to the martensite state. The temperature at which a shape memory material finishes transformation to the martensite state is known as the “martensite finish temperature.”

In one embodiment, the temporary correction template 10 can be formed of a shape memory material such as nitinol and can have a martensite start temperature that can be in the range of about 20 degrees to about 30 degrees Celsius less than the austenite start temperature, which can be in the range of about 0 degrees to about 10 degrees Celsius. The template 10 can have a martensite finish temperature that is about 0 degrees Celsius. In addition, the template 10 can have an austenite finish temperature that is below body temperature (37 degrees Celsius), for example, in the range of about 28 degrees to about 34 degrees Celsius, such that when the template reaches equilibrium with a temperature within the body, the shape memory material will exist in the fully austenitic state. A person skilled in the art will recognize that the transition temperatures between and within the martensite and austenite states may be selected to be any suitable temperature, depending on the composition of the shape memory material and/or the manufacturing process used to produce the shape memory material.

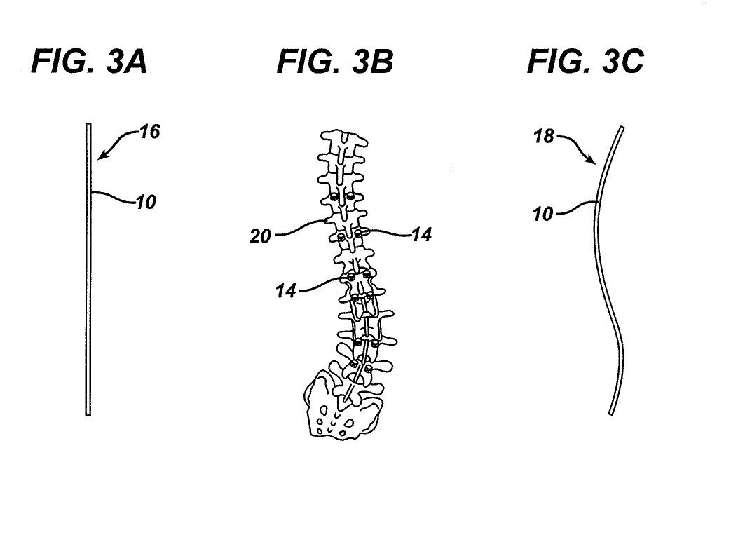

Referring now to both FIGS. 1A through 2, in general, the temporary correction template 10 can be formed to have any size and shape as needed for a particular spinal correction procedure. In one embodiment, the template 10 can be formed into an elongate member having a rod-like configuration. The template 10 can have any cross-sectional shape known in the art, including but not limited to, a circular cross-section, a polygonal-shaped cross-section, etc. In one particular embodiment, the template 10 can have a square cross-section. In other embodiments, the template 10 can have a square diamond shape. One example of such a shape is shown in FIG. 2, in which a retaining portion 12 of a spinal anchor includes a square diamond shape template 10 positioned therein. The cross-sectional dimension can have any size as needed, as will be appreciated by those skilled in the art. In general, the cross-sectional dimension can conform to a dimension of a rod receiving portion of a bone screw or bone anchor that can receive and hold the template 10 to aid in the correction. While the dimensions of the template can vary widely, in one embodiment, the template 10 can have a cross-sectional dimension with a length, width, or diameter that is in the range of about 3 mm to about 7 mm. In other embodiments, a length, width, or diameter can be in the range of about 1 mm or less to about 10 mm or more.

The correction template 10 can also have any length required for a particular corrective procedure. For example, in one embodiment, the template 10 can have a length effective to correct two spinal levels. In other embodiments, the template 10 can have a length effective to correct up to at least twelve spinal levels. A person skilled in the art will appreciate, therefore, that the template 10 can have any length effective to span between about two and about twelve levels. Alternatively or in addition, a plurality of templates 10 can be used to achieve a desired length. The plurality of templates 10 can each have the same length or can each have different lengths as needed in a particular corrective procedure. For example, if a template 10 is effective to span between two levels , then six templates 10 can be used together to span between twelve levels. Similar combinations of templates 10 will be appreciated by those of skill in the art.

The template 10 can generally have any shape in the martensitic state, or first configuration, as needed to fit a particular spinal deformity or injury. Generally, the template 10 can be curved and manipulated as needed such that it can be positioned to substantially conform parallel to the shape of the deformed or injured spine. In the case of a correction template 10 to be used for treating and/or correcting scoliosis, for example, the template 10 can be curved laterally or side to side to conform to a required curvature of a deformed spine afflicted with scoliosis. The template 10 can also be formed to have a spiral or a curve and spiral combination as needed to correspond to a scoliostic spinal rotation. In other embodiments in which the correction template 10 can be used to treat and/or correct spondylolisthesis, the template 10 can have a martensitic curve corresponding to an anterior/posterior spinal deformation. In still other embodiments in which the template 10 can be used to treat a spinal injury, such as a compression fracture, the template 10 can be formed into the corresponding shape as necessary. A person skilled in the art will appreciate the variety of lengths, shapes, and curvatures that the first configuration or martensitic configuration can take to conform to a particular spinal deformity or injury.

The template 10 can generally have any shape in the austenitic state, or second configuration, as needed to correspond to a desired, corrected, and/or natural lordotic shape of a deformed spine. For example, the curvature in the austenitic state or second configuration can be a shape that is corrected from a deformed curvature, but that is not necessarily a natural lordotic shape. The shape of the second configuration can depend on the amount of correction desired or required and may be medically limited by the amount of physical correction a particular spine can receive. The curvature in the austenitic state or second configuration can also be a natural lordotic shape. In some embodiments, it may be desirable to make an adjustment away from a natural lordotic state. Accordingly, in the martensitic state or first configuration, the template 10 may have a natural lordotic shape that will deform to a desired shape in the austenitic state or second configuration. As will be appreciated by those skilled in the art, the appropriate correctional forces applicable to a particular procedure can be determined by a physician, surgeon, technician, or other person skilled in the art.

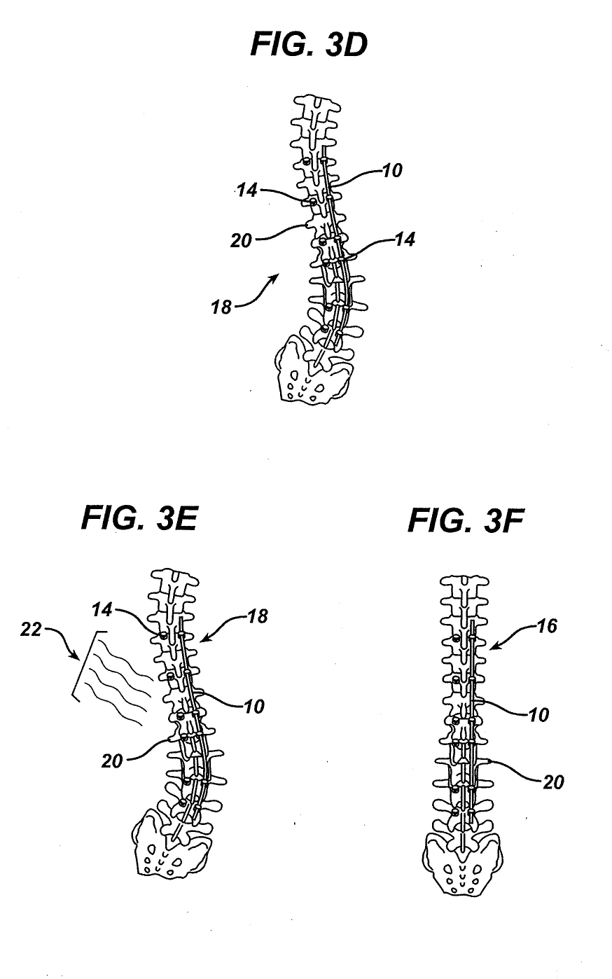

Referring now to FIGS. 3A-3F, methods generally associated with the use of the spinal correction template 10 will now be discussed. In general, the template 10 can be provided in the austenitic state, or second configuration 16, with a shape corresponding to a desired, corrected, or normal curvature of a spine, as shown in FIG. 3A. Any surgical approach known in the art can be used for the procedure, but in one embodiment, the approach used can be a standard posterior midline incision. In other embodiments, minimally invasive surgical techniques can be used, as will be described below. In another embodiment, conventional fluoroscopy-based pedicle screw insertion can be used to insert bone screws, pedicle screws, and/or bone anchors 14 into spinal vertebrae pedicles on two corresponding sides of a patient's spine 20, as is typically done for spinal corrective procedures as shown in FIG. 3B. In one embodiment, the screw insertion levels can be distributed, for example, from T3 to L4.

As will be appreciated by those skilled in the art, any spinal anchors 14 can be used, for example, hooks, bolts, wires, monoaxial pedicle screws and/or polyaxial pedicle screws. In one embodiment, the spinal anchors 14 can have U-shaped receiver members that are adapted to seat the template 10 and/or other spinal correction rods therein. In exemplary embodiments, appropriate anchor 14 diameters can include, but are not limited to, 4.5 mm, 5.0 mm, 6.0 mm, and 6.5 mm. As will further be appreciated by those skilled in the art, the bone anchors 14 can be attached to the spine 20 at any time before or after a temperature change is activated in the template 10, as will be described below.

In one exemplary method, the temperature of the template 10 can be changed and/or lowered to a martensite start temperature such that the template 10 can begin to become malleable and/or deformable. The temperature can continue to be lowered until the template 10 reaches the martensite finish temperature and the first configuration 18, at which point the template 10 is fully malleable and deformable. Any technique or mechanism known in the art can be used to lower the temperature of the template 10. For example, in one embodiment, the template can be cooled in an asepsis bath. In other embodiments, the template 10 can be placed in a freezer or exposed to a lowered ambient temperature such that its temperature is correspondingly lowered. Alternatively, an external cooling device may be used to transition the template 10 to a martensitic state. For example, a cold gas, such as liquid nitrogen or dry ice (CO₂), or other coolant may be applied directly or indirectly to the template 10. In other embodiments, the temperature can be lowered using a chilled saline or thermoelectric cooling device. As noted above, in one embodiment, the template 10 can be lowered to a temperature of about 0 degrees Celsius, though a person skilled in the art will appreciate that the martensite finish temperature can be adjusted as needed during manufacturing of the shape memory material. Other methods for lowering the temperature of a shape memory material will be appreciated by those skilled in the art, some of which can be found in U.S. Patent Application Publication No. 2007/0191831, entitled, “System and Method for Cooling a Spinal Correction Device Comprising a Shape Memory Material for Corrective Spinal Surgery,” which is incorporated herein by reference in its entirety.

Once the template 10 is fully malleable, it can be deformed to a first configuration 18 having any of the required curvatures as described above and as shown in FIG. 3C, such that it corresponds to the curvature of the deformed spine 20 shown in FIG. 3B. Any techniques known in the art can be used to determine the required curvature of the first configuration 18 for the template 10. In one embodiment, a soft mold rod can be inserted into the anchors 14 to acquire the required curvature. The template 10 can then be formed into a shape corresponding to the shape acquired by the soft mold rod. In other embodiments, the curvature of the deformed spine can be measured such that the template 10 can be molded to have the measured curvature. Such a measurement can be a physical measurement performed on the patient by a technician or surgeon. In other embodiments, scans can be made of the patient's body and measurements can be taken from the scans. For example, x-rays, magnetic resonance imaging (MRI) scans, computerized axial tomography (CAT) scans, etc. can be used to obtain images of a patient's spine so that measurements can be taken for determining the various curvatures to be used with the template 10.

Any techniques known in the art can be used to deform the template 10 to a first configuration 18. For example, a machine can be used to achieve the required curvatures and/or a physician, technician, or surgeon can form the required shape using tools, such as rod benders, as needed. The curved template 10 in the martensitic state can be placed adjacent to the spine 20 along one set of the anchors 14 such that it substantially conforms to the shape of the spine 20. In one embodiment, the incision can be filled with ice, cooled saline, or another cooling mechanism to control the local temperature during insertion of the template 10, thereby ensuring the template 10 remains malleable throughout the insertion process. The template 10 can be seated within the U-shaped receiver members of the anchors 14, as is known, such that the anchors 14 can secure the template 10 in a loose connection. In particular, a top anchor lock can be kept loose to enable movement of the template 10 with respect to the top anchor during the corrective process.

While there are various techniques for achieving correction of the spine 20, in one embodiment, the temperature of the template 10 can be changed and/or increased to an austenite start temperature such that the template 10 begins to “remember” the austenitic shape or the second configuration 16. The template 10 can begin to achieve the second configuration 16 by changing its shape, as shown in FIG. 3E. As the template 10 begins to change its shape, it can cause the spine 20 to also change shape in a way similar to the template 10 as the anchors 14 embedded in the spine 20 move with the template 10. In effect, the anchors 14 can at least partially restrain or prevent the shape recovery of the template 10 such that the template 10 generates significant shape recovery forces against the anchors 14. By using properly positioned anchors 14, the anchors 14 can transfer these corrective forces to the spine 20 such that the forces generated by the template 10 correct the shape of the spine 20.

In one embodiment, the temperature of the template 10 can continue to be increased to the austenite finish temperature at which point the template 10 is in its rigid, second configuration 16 corresponding to the desired, corrected, and or natural lordotic shape of the spine 20, as shown in FIG. 3F. As the template 10 achieves the second configuration 16, the spine 20 can be moved such that it also achieves a shape corresponding to the second configuration 16 and thereby achieves a desired, corrected, and/or natural lordotic shape. The correction can happen over any period of time as required, and as will be appreciated in the art. For example, the correction can require anywhere from about two minutes to about ten minutes, although correction can be achieved in shorter and/or longer time periods if needed.

Any mechanism 22 known in the art can be used to raise the temperature of the template 10 to its austenitic start and finish temperatures. For example, warm saline and/or water having a temperature in the range, for example, of about 50 degrees Celsius to about 60 degrees Celsius can be applied to the template 10. In other embodiments, the temperature of the template 10 can be raised using ambient air temperature. In one embodiment, the temperature increase can be derived solely from body temperature, or by a combination of body temperature and one or more other heat sources. In other embodiments, a high frequency, low voltage electric current can be used to raise the temperature. In still other embodiments, a heated liquid that circulates near or in contact with the template 10 can be used to raise the temperature. Alternatively, a heater can employ induction heating, resistance heating, electromagnetic radiation heating and/or any other suitable means for increasing the temperature of the shape memory material. As noted above, in one embodiment, the temperature of the template 10 can be raised to an austenitic start temperature in the range of about 28 degrees to about 34 degrees Celsius. The austenitic finish temperature, and the second configuration 16, can occur at about 37 degrees Celsius or body temperature.

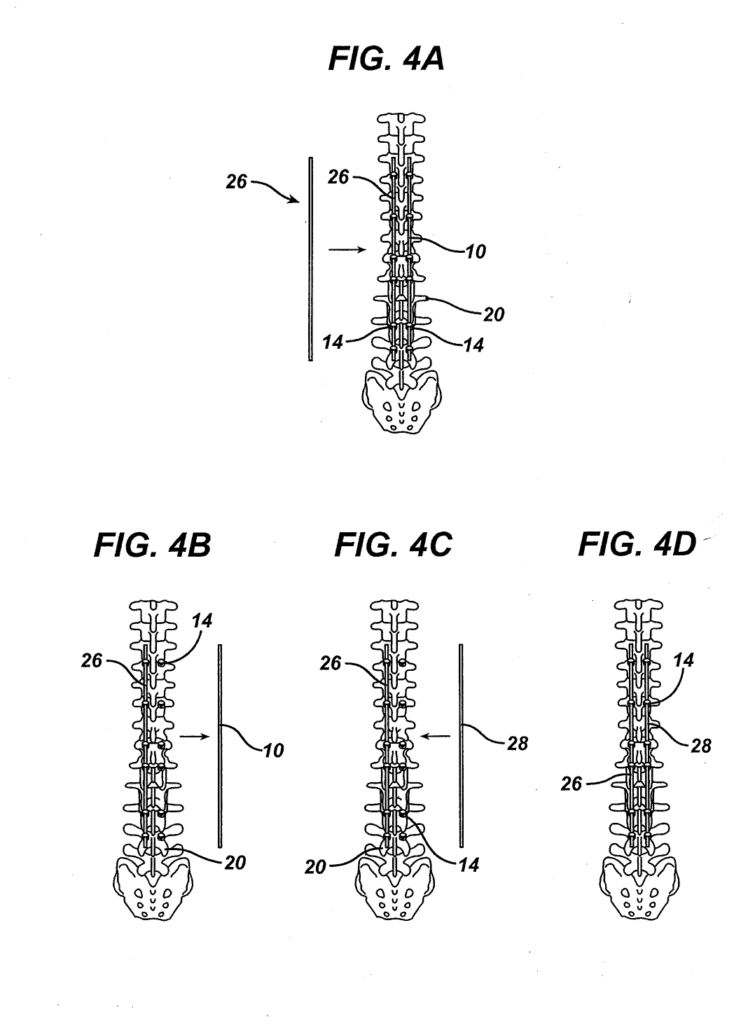

Referring now to FIGS. 4A-4D, a permanent primary spinal rod 26 can be attached to the spine at a position on an opposite side of and substantially parallel to the template 10. In one embodiment, the primary spinal rod 26 can have a shape corresponding to the first configuration 18 of the template 10 as shown in FIG. 4A, which is a shape corresponding to a desired corrected shape of the spine. The primary spinal rod 26 can be placed into the anchors 14 and secured rigidly and tightly thereto, as is typical. The template 10 can be removed from the anchors 14 and its attachment with the spine 20, as shown in FIG. 4B. A permanent secondary spinal rod 28 can replace the temporary correction template 10 and can be attached rigidly and tightly to the spine 20 using the anchors 14 as shown in FIGS. 4C and 4D. A person skilled in the art will appreciate that a single permanent rod can be used if desired. A person skilled in the art will also appreciate that the permanent spinal rods can be formed of any suitable biocompatible material known in the art, such as stainless steel, titanium, etc.

It is contemplated that the template 10 can also be used as a permanent rod and/or as one of two permanent rods. In some embodiments, it may be desirable to use two templates 10 in parallel to achieve correction of the spines The two templates 10 can be used simultaneously, particularly in procedures requiring significant adjustment. The two templates 10 can also be used in sequence, particularly in procedures in which adjustment is required along different axes. In some embodiments, the two parallel templates 10 can be replaced by one or two permanent rods as described above. In other embodiments, the two parallel templates 10 can become permanent rods and remain within the body during healing and/or fusion.

It will appreciated by those skilled in the art that once the necessary correction has been achieved, other procedures can be performed as needed. For example, adjustments can be made to the permanent rods and anchors to attain a proper balance. In some embodiments, facet decortication can be performed such that facet fusion can occur through the use of any fusion mechanism known in the art. In one embodiment, an autogenous bone graft harvested from the posterior iliac crest can be used to accomplish fusion.

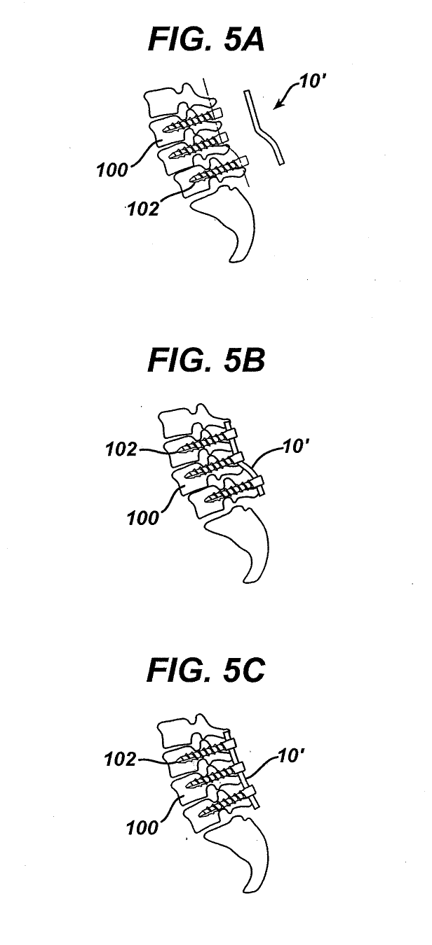

Referring now to FIGS. 5A-5C, one exemplary method for treating and/or correcting spondylolisthesis is provided. As shown in FIG. 5A, a temporary correction template 10′ is provided in the martensitic state with a shape substantially corresponding to a curvature of a deformed spine 100. Anchors 102 can be positioned within the vertebrae of the spine for receiving the template 10′. The template 10′ can be placed within the anchors 102 and loosely secured thereto such that the anchors 102 allow the template 10′ to move a small amount while changing shape, as shown in FIG. 5B. A temperature change can be induced in the template 10′, as described above, such that the template 10′ achieves an austenitic state in which it has a straightened and more normal configuration, as shown in FIG. 5C. The anchors 102 can transfer the correctional forces from the template 10′ to the spine and as such, a correction can be achieved. As noted above, one or more permanent rods can be placed into the anchors 102 as desired, and the temporary template 10′ can be removed after placement of a first permanent rod. As will be appreciated by those skilled in the art, there are various other devices and methods related to utilizing a shape memory material as an implant to correct spondylolisthesis, examples of which are described in U.S. Application No. 2007/0173828, entitled, “Spondylolistheses Correction System and Method of Correcting Spondylolistheses,” which is incorporated herein by reference in its entirety.

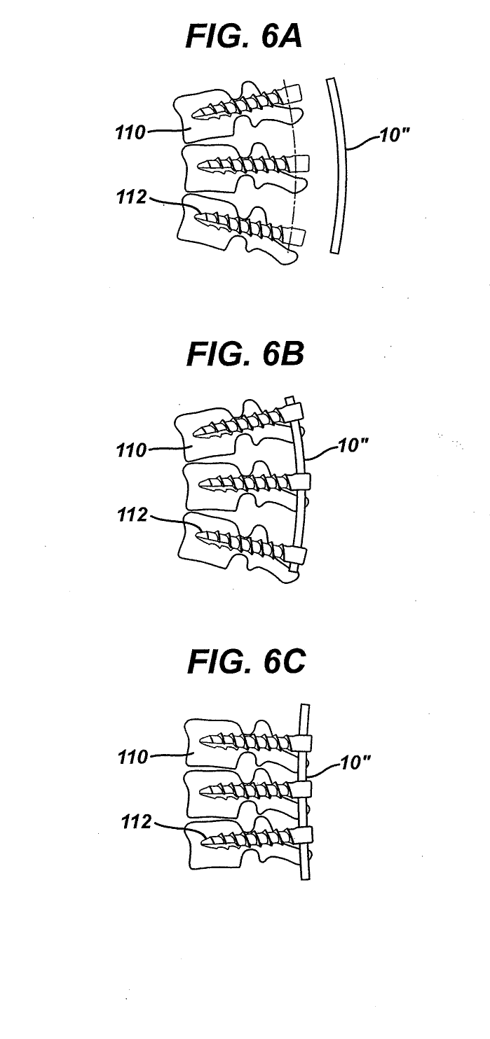

Referring now to FIGS. 6A-6C, one exemplary method for treating and/or correcting a spinal injury such as a compression fracture will now be discussed. As shown in FIG. 6A, a temporary correction template 10″ is provided in the martensitic state with a shape substantially corresponding to a curvature of an injured spine 110. Anchors 112 can be positioned as needed within the vertebrae of the spine 110 for receiving the template 10″. The template 10″ is then placed within the anchors 112 and loosely secured thereto such that the anchors 112 allow the template 10″ to move a small amount while changing shape, as shown in FIG. 6B. A temperature change can be induced in the template 10″, as described above, such that the template 10″ achieves an austenitic state in which it straightens to pull the vertebra apart and correct the compression fracture, as shown in FIG. 6C. The anchors 112 can transfer the correctional forces from the template 10″ to the spine and as such, a correction can be achieved. As noted above, one or more permanent rods can be placed into the anchors 112 as desired. The temporary template 10″ can be removed.

It is understood that all statements and characteristics applied to template 10 also apply equally to templates 10′ and 10″. The template 10 can have many configurations and in some embodiments, isolated segments of the template 10 may be controllably transitioned between the martensitic state and the austenitic state at a time. The template 10 can have insulation between different segments of the template 10 to prevent heat from transmitting from one segment to another to facilitate segmental correction. In another embodiment, the entire template 10 can transmit heat, so that heating a portion of the template 10 transmits heat to other portions of the template 10 to transition the entire template 10 to an austenitic state.

In other embodiments, the method of correcting a spinal defect using the template 10 can employ in vivo cooling of the template 10 to control the corrective forces applied to the spine. For example, if excessive or damaging force is applied to the spine during transition to the austenitic state, a cooling device may selectively and controllably cool the template 10 to remove some of the corrective force and loosen the spine. Then, heating may be subsequently controllably applied to reapply the corrective forces to the spine. Alternatively, after releasing the spine by in vivo cooling, additional anchoring devices may be inserted and connected to the template 10 to spread the load of the corrective forces over a greater area to reduce damage.

In one embodiment, the template 10 can include a feedback mechanism to allow for control of the heating and/or cooling and/or other transition-inducing parameter. For example, a sensor can measure the temperature of the template 10 and can send feedback to a monitor. The monitor can be used to adjust the heating and/or cooling of the template 10 to control the transition between states. The heating and/or cooling rate can be increased and decreased using the monitor. The sensor can also monitor stress rates and load on the anchoring devices to ensure the corrective forces stay within tolerable limits. Should stress rates and load surpass a tolerable limit, the sensor can indicate to the monitor to slow the heating and/or cooling process and/or to halt the process altogether. The monitor can be manually controlled by a technician and/or surgeon and/or can be remotely controlled via wired or wireless communication from an external controller.

In some embodiments, minimally invasive surgical techniques can be used to insert the template 10, as well as the permanent spinal rods 26, 28. Minimally invasive techniques can be particularly advantageous in that they can be achieved using one or more minimally invasive percutaneous incisions for accessing the spinal column. Such incisions minimize damage to intervening tissues, and thus reduce recovery time and post-operative pain. Such techniques can also eliminate the need to create a large working area at the surgical site.

In general, minimally invasive techniques can involve advancing the template 10 and/or the permanent rods 26, 28 in a lengthwise orientation along a minimally invasive pathway that extends from a minimally invasive percutaneous incision to a spinal anchor site. In an exemplary embodiment, a percutaneous access device can be used to create the minimally invasive pathway for receiving the template 10 and the rods 26, 28 and for delivering the template 10 and the rods 26, 28 to a spinal anchor site. The template 10 and the rods 26, 28 can be inserted through a lumen in the percutaneous access device in a lengthwise orientation, such that the template 10 and the rods 26, 28 are oriented substantially parallel to a longitudinal axis of the percutaneous access device. As the template 10 and/or the rods 26, 28 approach or reach the distal end of the pathway, the template 10 and/or the rods 26, 28 can be manipulated to orient them at a desired angle with respect to the percutaneous access device, preferably such that the template 10 and/or the rods 26, 28 are substantially parallel to the patient's spinal column. The template 10 and/or the rods 26, 28 can then optionally be positioned to couple, either directly or indirectly, to one or more spinal anchors. A fastening element or other closure mechanism, if necessary, can then be introduced into the spinal anchor site to fixedly mate the template 10 and/or the rods 26, 28 to the anchor(s).

In other embodiments, minimally invasive methods and devices are provided that can span multiple levels of the spine (e.g., three levels or more) while minimizing any associated tissue damage. For instance, methods can include delivering a plurality of percutaneous access devices to various spinal locations via a corresponding number of incisions (e.g., four percutaneous access devices would require four incisions). Next, a spinal fixation element (e.g., a correction template 10 or permanent spinal rod 26, 28) can be delivered through tissue via one of the existing incisions such that, for example, the spinal fixation element can be positioned adjacent an outer portion of a first percutaneous access device and delivered through the first incision into the patient. Once so positioned, the fixation element can be manipulated (e.g., via a manipulation instrument) so as to pass laterally through opposed sidewall openings of the first percutaneous access device and subsequently through the remainder of the access devices via similarly positioned opposed sidewall openings of each access device. Thus, the fixation element passes from the outside of the first percutaneous access device and transversely through the remainder of the devices. Such a delivery trajectory eliminates the need to introduce the fixation element axially through a proximal opening of the first percutaneous access device. Techniques and devices related to such minimally invasive techniques for spinal correction are known in the art and can be found, for example, in the Viper™ and Viper™2 Systems, manufactured and sold by Depuy Spine, Inc. of Raynham, Mass.

In some embodiments, a spinal correction kit can be provided and can include a plurality of spinal anchors configured to be positioned within bone, each spinal anchor having a rod receiving portion. The kit can also include one or more spinal correction template configured to be positioned within the rod receiving portions in an initial orientation parallel with a patient's spine and to move the patient's spine to a desired orientation in response to an activation energy. The kit can further include spinal correction template bending tool for changing a shape of the spinal correction template and one or more permanent spinal rods. In some embodiments, the spinal correction template can be a nitinol rod and the activation energy can be a change in temperature. The kit can also optionally include heating and/or cooling elements for applying heating and/or cooling to the spinal correction templates. In addition, a kit can include a monitor configured to apply, monitor, and/or adjust a temperature of the spinal correction template.

The exemplary systems, methods, and devices presented herein of correcting spinal deformities in a patient using a temporary correction template formed from a shape memory material provides significant advantages over prior spinal correction systems. The forces required to move the displaced vertebra can be carefully controlled and are focused closer to the spine, potentially reducing the overload to the spine or implants. The systems and methods disclosed herein can reduce operating costs, operation time, and tissue damage and blood loss to the patient while achieving desired and necessary spinal corrections.

One skilled in the art will appreciate further features and advantages of the presently disclosed methods and/or devices based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. A method of correcting a spinal deformity, comprising:
attaching to at least a portion of a patient's spine a spinal correction template having a first configuration;
activating the spinal correction template such that the template achieves a second configuration to cause the spine to assume an orientation substantially corresponding to the second configuration of the spinal correction template;
attaching a primary spinal rod to at least a portion of spine; and
removing the spinal correction template.

2. The method of claim 1, further comprising inserting a secondary spinal rod in place of the spinal correction template.

3. The method of claim 1, wherein the spinal correction template is formed of a shape-memory alloy.

4. The method of claim 3, wherein the shape-memory alloy comprises nitinol.

5. The method of claim 1, wherein the step of activating the spinal correction template includes heating the spinal correction template to a temperature above an activation temperature of the spinal correction template.

6. The method of claim 1, wherein the primary spinal rod has a configuration substantially the same as the second configuration of the spinal correction template.

7. The method of claim 2, wherein the secondary spinal rod has a configuration substantially the same as the second configuration of the spinal template.

8. The method of claim 1, wherein the first configuration of the spinal correction template substantially corresponds to the uncorrected shape of a deformed spine.

9. The method of claim 1, wherein the second configuration of the spinal correction template substantially corresponds to the shape of a normal spine.

10. The method of claim 1, wherein the spinal correction template has a length effective to provide a twelve-level spinal correction.

11. The method of claim 1, wherein the spinal correction template has a length effective to provide at least a three-level spinal correction.

12. The method of claim 1, wherein the step of placing the spinal correction template into the body is performed through a minimally invasive surgical technique.

13. The method of claim 1, wherein the spinal correction template is attached to the spine at a temperature less than a temperature at which activation occurs.

14. The method of claim 1, wherein the step of activating occurs at a temperature in the range of about 28 degrees Celsius to about 37 degrees Celsius.

15. The method of claim 1, wherein the step of attaching occurs when the spinal correction template is at a temperature of about 0 degrees Celsius.

16. The method of claim 1, wherein heating the spinal correction template comprises transferring heat from a patient's body.

17. The method of claim 1, wherein heating the spinal correction template includes one of radiant heating, liquid heating, and electromagnetic heating.

18. A spinal correction kit, comprising:
a plurality of spinal anchors configured to be positioned within bone, each spinal anchor having a rod receiving portion; and
at least one spinal correction template configured to be positioned within the rod receiving portions in an initial orientation parallel with a patient's spine and to move the patient's spine to a desired orientation in response to an activation energy.

19. The kit of claim 18, further comprising at least one permanent spinal rod.

20. The kit of claim 18, wherein the spinal correction template comprises a nitinol rod and the activation energy comprises a change in temperature.
